(19) United States
(12) Patent Application Publication
(10) Pub. No.: US 2024/0249846 A1
(43) Pub. Date: Jul. 25, 2024

(54) FEEDBACK-BASED INTERVENTION SYSTEM FOR MANAGING INDICATION AND OPERATING METHOD THEREOF

(71) Applicant: ODN CO., LTD., Seoul (KR)

(72) Inventors: Sang Youl RHEE, Seoul (KR); Sung Young LEE, Seongnam-si (KR); Hafiz Syed Muhammad BILAL, Suwon-si (KR)

(73) Assignee: ODN CO., LTD., Seoul (KR)

(21) Appl. No.: 18/292,144

(22) PCT Filed: Dec. 29, 2021
(86) PCT No.: PCT/KR2021/020120

§ 371 Date: Jan. 25, 2024

(30) Foreign Application Priority Data
Sep. 16, 2021 (KR) 10-2021-0124103

(51) Int. Cl. G16H 50/30; G16H 10/60; G16H 20/10; G16H 20/30; G16H 20/40; G16H 20/60; G16H 50/20

(52) U.S. Cl. CPC G16H 50/30; G16H 10/60; G16H 20/10; G16H 20/30; G16H 20/40; G16H 20/60; G16H 50/20

(57) ABSTRACT

A feedback-based intervention system includes a data collector configured to collect profile data and life log data of a user, a user data quantification unit configured to extract at least one quantification factor through an evaluation process of the user based on the collected profile data, and to quantify behavior data of the collected life log data, which corresponds to the extracted quantification factor, an intervention information controller configured to determine a health status of the user based on the quantified behavior data, and to generate at least one piece of intervention information based on a determination result of the health status, and a communication unit configured to provide the generated intervention information to the user, wherein the intervention information controller receives feedback information of the user, corresponding to the provided intervention information, and adjusts a provision schedule of the intervention information based on the received feedback information.

### CROSS-REFERENCE TO RELATED APPLICATION

This application claims priority to and the benefit of Korean Patent Application No. 10-2021-0124103, filed on Sep. 16, 2021, the disclosure of which is incorporated herein by reference in its entirety.

### BACKGROUND OF THE INVENTION

### 1. Field of the Invention

The present invention relates to a feedback-based intervention system and an operating method thereof, and more particularly to a technical aspect for optimizing intervention information for managing indication based on feedback of a user.

### 2. Description of the Related Art

Recently, as a mobile device has been more common, the number of users who use a smart obesity management service based on a smart phone, a wearable device, a smart scale, and an application program has increased.

In detail, an existing smart obesity management service provides a user with information necessary to reach a goal set by the user based on various basic information indicating the user's current status (a body mass index (BMI), a current obesity rate, etc.) but has a limitation in that user feedback is not properly applied to the provided information and an optimized service is not provided to the user.

### CITED REFERENCE

### Patent Document

Korean Patent Publication No. 10-2016-0139960, “Web-based obesity management system”
Korean Patent No. 10-1315907, “Exercise prescription system and exercise prescription method”

### SUMMARY OF THE INVENTION

Therefore, the present invention has been made in view of the above problems, and it is an object of the present invention to provide an intervention system for providing a user-customized indication management service and an operating method thereof based on basic information of a user and behavior data based on a lifestyle of the user.

It is another object of the present invention to provide an intervention system for providing a more effective and optimized indication management service to a user by applying user feedback for a provided indication management service and an operating method of the intervention system.

In accordance with an aspect of the present invention, the above and other objects can be accomplished by the provision of a feedback-based intervention system comprising: a data collector configured to collect profile data and life log data of a user; a user data quantification unit configured to extract at least one quantification factor through an evaluation process of the user based on the collected profile data, and to quantify behavior data of the collected life log data, which corresponds to the extracted quantification factor; an intervention information controller configured to determine a health status of the user based on the quantified behavior data, and to generate at least one piece of intervention information based on a determination result of the health status; and a communication unit configured to provide the generated intervention information to the user, wherein the intervention information controller receives feedback information of the user, corresponding to the provided intervention information, and adjusts a provision schedule of the intervention information based on the received feedback information.

In accordance with an aspect, the profile data may include at least one of gender information, age information, initial height information, initial weight information, or disease history information of the user.

In accordance with an aspect, the life log data may include at least one of physical activity information, weight information, height information, blood pressure information, drinking information, smoking information, diet information, or meal information of the user, which is collected for a preset collection time.

In accordance with an aspect, the user data quantification unit may calculate at least one piece of initial biological information of an initial obesity class, an initial body mass index (BMI), an initial calorie consumption, or an initial exercise amount of the user based on the collected profile data and may extract a quantification vector based on the calculated initial biological information.

In accordance with an aspect, the user data quantification unit may accumulate behavior data corresponding to the extracted quantification factor of the collected life log data during a preset accumulation time and may quantify the accumulated behavior data based on a quantification rule preset by an expert.

In accordance with an aspect, the user data quantification unit may quantify the accumulated behavior data by calculating basal metabolic rate (BMR) information of the user based on the collected life log data and performing an operation on the calculated BMR information and an activity index corresponding to the accumulated behavior data.

In accordance with an aspect, the quantification factor may be a factor corresponding to at least one of meal information, physical activity information, smoking information, or drinking information of the user.

In accordance with an aspect, the intervention information controller may determine the quantified behavior data as at least one health status of ‘Healthy’, ‘Normal’, or ‘Risk’ based on a preset classification threshold, may calculate behavior change information of the user based on the collected life log data, and may calculate the at least one piece of intervention information based on a determination result of the health status and the calculated behavior change information.

In accordance with an aspect, the intervention information controller may monitor biosignal information of the user, corresponding to the calculated behavior change information, and may determine whether the generated intervention information is provided based on a monitoring result of the biosignal information.

In accordance with an aspect, the communication unit may determine a provision schedule of the generated intervention information based on at least one of an eating habit of the user, an exercise routine of the user, an achievement goal of the user, preference of the user, or prescription of an expert.

In accordance with an aspect, the intervention information controller may determine a number of times the intervention information is provided and a period at which the intervention information is provided based on a difference between a body mass index (BMI) corresponding to the collected feedback information and a BMI corresponding to a time at which the generated intervention information is provided to the user.

In accordance with another aspect of the present invention, there is provided an operating method of a feedback-based intervention system, the method comprising: collecting profile data and life log data of a user, by a data collector; extracting at least one quantification factor through an evaluation process based on the collected profile data and quantifying data of the collected life log data, which corresponds to the extracted quantification factor, by a user data quantification unit; determining a health status of the user based on the quantified behavior data and generating at least one piece of intervention information based on a determination result of the health status, by an intervention information controller; providing the generated intervention information to the user, by a communication unit; and receiving feedback information of the user, corresponding to the provided intervention information, and adjusting a provision schedule of the intervention information based on the received feedback information, by the intervention information controller.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a diagram for explaining a feedback-based intervention system according to an embodiment;

FIG. 2 is a diagram for explaining a user data quantification unit according to an embodiment;

FIG. 3 is a diagram for explaining an intervention information controller according to an embodiment in detail;

FIG. 4 is a diagram for explaining an example of collection of user information in a feedback-based intervention system according to an embodiment;

FIGS. 5 and 6 are diagrams for explaining an example of intervention information provided in a feedback-based intervention system according to an embodiment; and

FIG. 7 is a diagram for explaining an operating method of a feedback-based intervention system according to an embodiment.

### DETAILED DESCRIPTION OF THE INVENTION

Specific structural and functional descriptions of embodiments according to the concept of the present disclosure disclosed herein are merely illustrative for the purpose of explaining the embodiments according to the concept of the present disclosure. Furthermore, the embodiments according to the concept of the present disclosure can be implemented in various forms and the present disclosure is not limited to the embodiments described herein.

The embodiments according to the concept of the present disclosure may be implemented in various forms as various modifications may be made. The embodiments will be described in detail herein with reference to the drawings. However, it should be understood that the present disclosure is not limited to the embodiments according to the concept of the present disclosure, but includes changes, equivalents, or alternatives falling within the spirit and scope of the present disclosure.

The terms such as “first” and “second” are used herein merely to describe a variety of constituent elements, but the constituent elements are not limited by the terms. The terms are used only for the purpose of distinguishing one constituent element from another constituent element. For example, a first element may be termed a second element and a second element may be termed a first element without departing from the scope of rights according to the concept of the present invention.

It will be understood that when an element is referred to as being “on”, “connected to” or “coupled to” another element, it may be directly on, connected or coupled to the other element or intervening elements may be present. In contrast, when an element is referred to as being “directly on,” “directly connected to” or “directly coupled to” another element or layer, there are no intervening elements or layers present. Other words used to describe the relationship between elements should be interpreted in a like fashion (e.g., “between,” versus “directly between,” “adjacent,” versus “directly adjacent,” etc.).

The terms used in the present specification are used to explain a specific exemplary embodiment and not to limit the present inventive concept. Thus, the expression of singularity in the present specification includes the expression of plurality unless clearly specified otherwise in context. Also, terms such as “include” or “comprise” in the specification should be construed as denoting that a certain characteristic, number, step, operation, constituent element, component or a combination thereof exists and not as excluding the existence of or a possibility of an addition of one or more other characteristics, numbers, steps, operations, constituent elements, components or combinations thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The present disclosure will now be described more fully with reference to the accompanying drawings, in which exemplary embodiments of the invention are shown. This disclosure may, however, be embodied in many different forms and should not be construed as limited to the exemplary embodiments set forth herein. Like reference numerals in the drawings denote like elements.

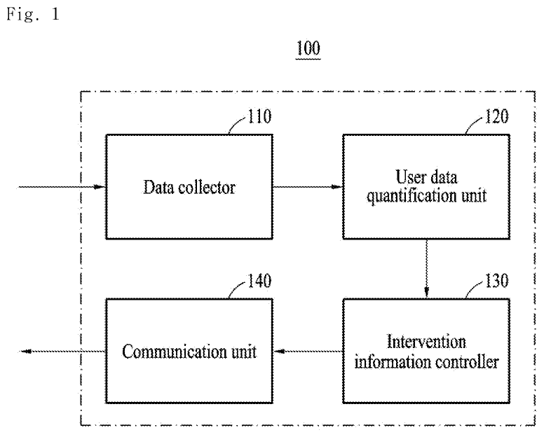

FIG. 1 is a diagram for explaining a feedback-based intervention system according to an embodiment.

Referring to FIG. 1, an intervention system 100 according to an embodiment may provide a user-customized indication management service based on basic information of a user and behavior data depending on a lifestyle of the user.

The intervention system 100 may provide a more effective and optimized indication management service to the user by applying user feedback for the provided indication management service.

In detail, the indication may include obesity, metabolic syndrome, pre-diabetes, diabetes, nonalcoholic fatty liver disease (NAFLD), and other diseases or symptoms that have been shown to improve medically using weight intervention, and the intervention system 100 may provide optimized intervention information for indication management to the user.

To this end, the intervention system 100 may include a data collector 110 for collecting data related to a lifestyle of the user, and a user data quantification unit 120 for quantifying the behavioral attributes of the user, acquired from the data of the user, according to a guideline/rule set by an expert to identify a behavior status of the user.

The intervention system 100 may include an intervention information controller 130 for determining a health status of the user based on the identified behavior status and generating intervention information based on the determination result, and a communication unit 140 for providing the generated intervention information to the user.

The data collector 110 according to an embodiment may collect profile data and life log data of the user.

According to an aspect, the data collector 110 may collect profile data and life log data based on response to a smart device for collecting information on the user and survey of the user, and in this case, the profile data may refer to data of the user, which is collected at the beginning of use of the intervention system 100, and the life log data may refer to data of the user, which is collected for a preset collection time, in detail, in real time.

For example, the profile data may include at least one of gender information, age information, initial height information, initial weight information, or initial disease history information of the user, and here, the disease history information may further include prescribing information of an expert. In addition, the profile data may further include target information (e.g., target obesity and/or weight information) of the user.

The life log data may include at least one of physical activity information, weight information, height information, blood pressure information, drinking information, smoking information, diet information, disease history information, or meal information of the user, which is collected for a preset collection time, and here, the physical activity information may include information on the type of exercise the user is performing, an exercise time, and the number of exercises, and the life log data may include biosignal information of the user that may be collected through a previously known smart device.

According to an aspect, the weight information, the height information, the blood pressure information, and the biosignal information of the life log data may also be collected in association with activities of the user, such as physical activity information, drinking information, smoking information, diet information, and meal information of the user. In other words, the data collector 110 may collect real-time weight information, height information, blood pressure information, and biosignal information of the user in exercise, as the life log data.

The user data quantification unit 120 according to an embodiment may extract at least one quantification factor through an evaluation process of the user based on the collected profile data and may quantify behavior data corresponding to the extracted quantification factor of the collected life log data.

According to an aspect, the user data quantification unit 120 may calculate at least one of initial biological information of initial obesity class, an initial body mass index (BMI), an initial calorie consumption, or an initial exercise amount of the user based on the collected profile data, and may extract a quantification vector based on the calculated initial biological information.

According to an aspect, the user data quantification unit 120 may extract a quantification vector based on the calculated initial biological information and pre-stored target information of the user, and here, the target information may be information pre-input by the user or information calculated through calculation based on the calculated initial biological information.

For example, the user data quantification unit 120 may calculate at least one piece of initial information of an initial obesity class, an initial BMI, an initial calorie consumption, and an initial exercise amount of the user using an already known method based on the profile data.

The user data quantification unit 120 may calculate a body mass index

$$B M I = \frac{Weight ( kg )}{{Height ( m )}^{2}}$$

based on the weight information and the height information of the user, and may determine an obesity class based on the calculated BMI.

For example, when the user is Korean, the user data quantification unit 120 may determine an obesity class of the user based on Table 1 below.

TABLE 1

| Obesity class | BMI (kg/m²) |
|---|---|
| Underweight | <18.5 |
| Normal | 18.5-22.9 |
| pre-obesity | 23-24.9 |
| stage 1 obesity (first class) | 25-29.9 |
| stage 2 obesity (second class) | 30-34.9 |
| stage 3 obesity (third class) | ≥35 |

In other words, when the calculated BMI satisfies 18≤BMI≤22.9, an obesity class of the user may be classified as a normal class, when the calculated BMI satisfies 23≤BMI≤29.9, the obesity class of the user may be classified as a first class, when the calculated BMI satisfies 30≤BMI≤34.9, the obesity class of the user may be classified as a second class, and when the calculated BMI satisfies 35≤BMI, the obesity class of the user may be classified as a third class.

When the user is not Korean, the user data quantification unit 120 may determine an obesity class of the user based on Table 2 below, and when the user is Asian, not Korean, the user data quantification unit 120 may determine the obesity class of the user based on at least one of Table 1 or 2.

TABLE 2

| obesity class(obesity class) | BMI (kg/m²) |
|---|---|
| Underweight | <18.5 |
| Normal | 18.5-24.9 |
| pre-obesity | 25.0-29.9 |
| stage 1 obesity(first class) | 30-34.9 |
| stage 2 obesity(second class) | 35.0-39.9 |
| stage 3 obesity(third class) | ≥40 |

According to an aspect, the user data quantification unit 120 may identify an activity domain (e.g., a domain related to physical activity, a domain related to diet, and a domain related to meal) of the user for providing intervention information corresponding to the determined obesity class and may extract a quantification factor corresponding to the identified activity domain. For example, the quantification factor may be a factor corresponding to at least one piece of information of meal information, physical activity information, smoking information, or drinking information of the user.

According to an aspect, the user data quantification unit 120 may accumulate behavior data corresponding to the extracted quantification factor from the collected life log data for a preset accumulation time and may quantify the accumulated behavior data based on a quantification rule preset by an expert.

In detail, the user data quantification unit 120 may quantify the accumulated behavior data by calculating basal metabolic rate (BMR) information of the user based on the collected life log data and performing an operation on the calculated BMR information and an activity index corresponding to the accumulated behavior data.

In more detail, the user data quantification unit 120 may calculate the basal metabolic rate (BMR) information shown in Equation 1 below based on the weight information and height information of the user, which are collected from the collected life log data.

$$B M R = 10 \times Weight ( kg ) + 6.25 \times Height ( cm ) - 5 \times Age ( yesrs ) + 5 ( man ) \qquad [ Equation 1 ]$$

$$B M R = 10 \times Weight ( kg ) + 6.25 \times Height ( cm ) - 5 \times Age ( yesrs ) - 161 ( woman )$$

Then, the user data quantification unit 120 may quantify the accumulated behavior data by applying an activity index corresponding to the physical activity information of the accumulated behavior data to the basal metabolic rate (BMR) information.

For example, based on the accumulated behavior data (physical activity information), the user data quantification unit 120 may perform quantification by applying an activity index ‘1.22’ to the basal metabolic rate (BMR) information (i.e., 1.22×BMR) when the user performs exercise 1 to 3 times a week and applying an activity index ‘1.373’ to the BMR information (i.e., 1.373×BMR) when the user performs exercise 3 to 5 times a week.

In addition, based on the accumulated data, the user data quantification unit 120 may perform quantification by applying an activity index ‘1.55’ to the basal metabolic rate (BMR) information (i.e., 1.55×BMR) when the user performs exercise 5 to 7 times a week and applying an activity index ‘1.95’ to the BMR information (i.e., 1.95×BMR) when the user performs exercise 7 times or more a week.

The user data quantification unit 120 may calculate a body mass index (BMI) based on the weight information and height information of the user, which are collected in real time from the collected life log data.

The intervention information controller 130 according to an embodiment may determine a health status of the user based on the behavior data quantified by the user data quantification unit 120 and may generate at least one piece of intervention information based on the determination result of the health status.

For example, the intervention information may include at least one piece of information of physical activity recommendation information, diet recommendation information, meal recommendation information, or clinic recommendation information. That is, the intervention information may refer to at least one piece of information of physical activity, diet, meal, or clinic, which is optimized for the user, and here, the activity information may include at least one piece of information of activity type, activity number, activity execution time, activity recommendation time, or activity recommendation cycle.

For example, clinic recommendation information may include at least one of clinic information based on expert prescription, drug clinic information, or surgery clinic information.

In detail, the clinic information based on expert prescription may be associated with at least one of a smart device or a medical device of the user to provide information helpful for chronic diseases and user health promotion, and in detail, the clinic information based on expert prescription may include information related to digital therapeutics.

The drug clinic information may include information on drug recommendations and drug-specific dose/usage for indication treatment/improvement.

According to an aspect, the intervention information controller 130 may provide drug recommendation information based on at least one of the body mass index (BMI) or the disease history information of the user.

For example, the intervention information controller 130 may provide drug recommendation information for obesity treatment when the obesity class derived from the BMI of the user is above a first class (stage 1 obesity) or in the case of pre-obesity and obesity-related complications (e.g., at least one disease of type 2 diabetes, high blood pressure, coronary artery disease, chronic kidney failure, liver dysfunction, glaucoma, or pancreatitis).

According to an aspect, the intervention information controller 130 may provide the drug recommendation information for recommending at least one drug among a plurality of drugs in consideration of complications of the user and severity (mild, moderate, and severe) information depending on the complications.

For example, the intervention information controller 130 may collect and store product description information of drugs based on a clinical trial in advance, may extract information on drugs except for a drug that cannot be used by the user among a plurality of drugs based on the stored product description information, and the complications and severity information of the user, and may provide recommendation information based on the extracted information.

According to an aspect, the intervention information controller 130 may also provide information on dosage/usage for each drug based on stored product description information.

For example, the intervention information controller 130 may extract a time at which the user takes a drug from at least one of the smart device or the medical device of the user and may provide information on the dosage/usage for each drug at a next time at which the user takes the drug, which is derived based on the extracted time at which the user takes the drug, a dosing cycle derived from the product description information, and taking time information (e.g., before meals/during meals/after meals, etc.).

According to an aspect, the intervention information controller 130 may determine the quantified behavior data as at least one health status of ‘Healthy’, ‘Normal’, or ‘Risk’ based on a preset classification threshold.

For example, based on the quantified behavior data, the intervention information controller 130 may determine the health status as ‘Healthy’ when the user performs exercise 6 times or more a week, may determine the health status as ‘Normal’ when the user performs exercise 3 to 6 times or more a week, and may determine the health status as ‘Risk’ when the user performs exercise less than 3 times a week.

Based on the quantified behavior data, the intervention information controller 130 may determine the health status as ‘Healthy’ when the user performs exercise for 180 minutes or more a week, may determine the health status as ‘Normal’ when the user performs exercise for 150 to 180 minutes a week, and may determine the health status as ‘Risk’ when the user performs exercise less than 150 minutes a week.

According to an aspect, the intervention information controller 130 may calculate behavior change information of the user based on the collected life log data and may calculate at least one piece of intervention information based on the determination result of the health status and the calculated behavior change information.

In detail, the intervention information controller 130 may provide information corresponding to the quantified behavior data of the user of the pre-stored initial intervention information to the user to induce behavior change, may monitor a behavior of the user based on the behavior change, and may determine whether the intensity of intervention information to be provided to the user is changed based on the monitoring result of the behavior change.

For example, the intervention information controller 130 may receive the quantified behavior data from the user data quantification unit 120 in real time, and may determine that a behavior of the user is changed when a difference between the quantified behavior data (i.e., quantified current behavior data) received after the behavior change is induced and the quantified behavior data received before the behavior change is induced is greater than a preset behavior change threshold.

According to an aspect, when determining that the difference of the quantified behavior data is between a first behavior threshold and a second behavior threshold (here, the second behavior threshold>the first behavior threshold) and there is little change in a lifestyle of the user, the intervention information controller 130 may increase a period of providing the same intervention information as the initial intervention information provided for inducing the behavior of the user.

When determining that the difference of the quantified behavior data is greater than the second behavior threshold and there is an appropriate change in lifestyle of the user, the intervention information controller 130 may provide intervention information of one level higher than the intensity of the initial intervention information provided for inducing the behavior of the user.

According to an aspect, the intervention information controller 130 may calculate at least one piece of intervention information based on the determination result of the health status, the calculated behavior change information, and the BMI received from the user data quantification unit 120.

For example, the intervention information controller 130 may calculate intervention information including information about the exercise, the number of exercises corresponding to the determination result of the health status, and the exercise intensity corresponding to the calculated behavior change information

When the BMI of the user is greater than 30, the intervention information controller 130 may also calculate intervention information including clinic information on at least one of a drug or a surgery.

In detail, for example, the intervention information controller 130 may calculate exercise information and/or meal information, which is capable of changing a class corresponding to the current obesity class to a class lower than the current obesity class as intervention information based on the calculated behavior change information.

The intervention information controller 130 may calculate exercise information and/or meal information corresponding to at least one of a preset target calorie consumption and target weight information calculated through a difference between the current BMI and the target BMI as intervention information.

According to an aspect, the intervention information controller 130 may monitor biosignal information of the user, corresponding to the calculated behavior change information, and may determine whether the generated intervention information is provided, based on the monitoring result of the biosignal information.

In detail, the intervention information controller 130 may analyze negative impact on the body of the user in response to provision of the generated intervention information based on the calculated behavior change information, and may stop providing the intervention information when the analysis result is determined to be negative.

According to an aspect, when stopping providing the intervention information, the intervention information controller 130 may re-generate intervention information by re-adjusting at least one of the type, number, or intensity of intervention.

The communication unit 140 according to an embodiment may provide the intervention information generated by the intervention information controller 130 to the user.

According to an aspect, the communication unit 140 may determine a provision schedule of the generated intervention information based on at least one of an eating habit of the user, an exercise routine of the user, an achievement goal of the user, preference of the user, or prescription of an expert.

In detail, the communication unit 140 may determine the eating habit and the exercise routine of the user based on the collected life log data and may determine the provision schedule of the intervention information for each user based on the determination result of the eating habit and the exercise routine of the user. In this case, the provision schedule may apply the time/standard according to the opinion of the expert.

The communication unit 140 may determine the provision schedule of the intervention information by applying preference that is previously input by the user, may determine the provision schedule of the intervention information based on at least one of recommendation or prescription of the expert, and may determine the provision schedule of the intervention information according to a preset cycle on a daily basis in response to a goal that the user needs to achieve in one day.

The intervention information controller 130 may receive feedback information of the user, corresponding to the provided intervention information, and may adjust the provision schedule of the intervention information based on the received feedback information.

For example, the data collector 110 may collect feedback information (life log data) of the user, corresponding to the provided intervention information, and the intervention information controller 130 may receive the feedback information from the data collector 110.

According to an aspect, the intervention information controller 130 may receive user feedback corresponding to the drug clinic information and may determine an effect of drug clinic information and whether a side effect occurs.

For example, when receiving the intervention information corresponding to the drug clinic information, the user may perform a behavior (e.g., taking medication) corresponding thereto, may monitor the behavior through at least one of the smart device or the medical device of the user, and may provide the monitoring result as feedback information.

The intervention information controller 130 may determine an effect of the drug clinic information and whether a side effect occurs based on at least one of a weight change history or a history of vomiting of the user, included in the user feedback corresponding to the drug clinic information.

In detail, for example, when the weight of the user is not reduced by more than a critical weight ratio (e.g., 5%) during a preset monitoring cycle, the intervention information controller 130 may determine that an effect of the provided drug clinic is not large. When the user vomits exceeding a threshold number of vomiting during a preset monitoring cycle, the intervention information controller 130 may determine that a side effect occurs according to the provided drug clinic.

According to an aspect, the intervention information controller 130 may re-generate intervention information in consideration of an effect of the drug clinic information and the determination result of whether a side effect occurs.

For example, when determining that the effect of the provided drug clinic is not large, the intervention information controller 130 may generate intervention information to which recommendation information and dosage/usage information on a different drug from the provided drug information is applied.

When determining that a side effect occurs due to the provided drug clinic, the intervention information controller 130 may also generate intervention information to which expert prescription is applied.

According to an aspect, the intervention information controller 130 may determine the number of times the intervention information is provided and a period at which the intervention information is provided based on a difference between a BMI corresponding to the collected feedback information and a BMI corresponding to a time of providing the generated intervention information to the user.

In detail, the intervention information controller 130 may receive the BMI corresponding to the feedback information and the BMI corresponding to the time of providing the intervention information to the user from the user data quantification unit 120, and may determine the number of times the intervention information is provided and a period at which the intervention information is provided based on a difference between the received BMIs.

For example, the intervention information controller 130 may increase the number of interventions and/or may reduce a period at which intervention is provided when the difference between the received BMIs is equal to or greater than a preset body mass threshold, may not change the number of interventions and/or the period at which intervention is provided when there is no difference between the received BMIs (i.e., when the difference is 0), and may reduce the number of interventions and/or may increase the period at which intervention is provided when the difference between the received BMIs is less than the body mass threshold.

According to an aspect, the intervention information controller 130 may adjust the provision schedule of the intervention information based on the difference between the received BMIs, and may optimize the provision schedule of the intervention information in consideration of health status, body requirements, and physical ability of the user, which are derived from the collected life log data.

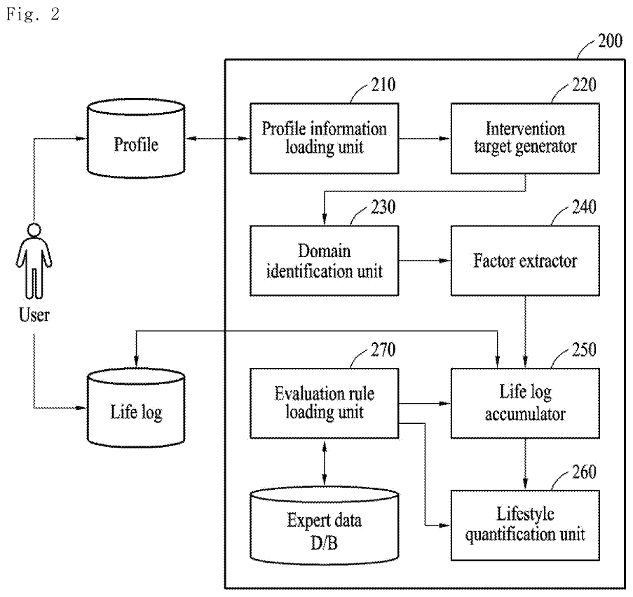

FIG. 2 is a diagram for explaining a user data quantification unit according to an embodiment.

Referring to FIG. 2, a data quantification unit 200 according to an embodiment may perform an intervention target evaluation process for estimating a target to be achieved by a user in order to improve a level of an activity status according to a lifestyle of the user, and a behavior quantification process for quantifying acquired behavior data related to a specific behavior from a life log.

In detail, the intervention target evaluation process may be performed through a profile information loading unit 210, an intervention target generator 220, a domain identification unit 230, and a factor extractor 240, and the behavior quantification process may be performed through an evaluation rule loading unit 240, a life log accumulator 250, and a lifestyle quantification unit 260.

The profile information loading unit 210 may receive profile information of the user in order to understand a situation of the user, and here, the profile information may include at least one of gender information, age information, initial height information, initial weight information, or disease history information of the user.

The intervention target generator 220 may calculate the initial biological information of the user for providing the intervention information through the evaluation process based on profile information of the user and a targeted activity based on a preset target.

For example, the intervention target generator 220 may calculate initial biological information including at least one of an obesity class (OC), a body mass index (BMI), a required calorie consumption (Cal), or a required exercise amount (phyexe), which are calculated using Equation 2 below.

$${T}_{O C} = {C}_{OC} - 1 \qquad [ Equation 2 ]$$

$${T}_{BMI} - {C}_{BMI} = Δ ( kg )$$

$${Week}_{Total} = Δ ( kg ) * 2$$

$${Cal}_{Rec} = {Cal}_{Req} - 500 ( day )$$

$${Rec}_{{Phy}_{exe}} = ( {Rec}_{Steps} - {C}_{steps} ) * ( 0.33 ) * ( Week )$$

Here, T is a target value, C is a current value, Cal is calorie, Rec is recommendations, and Req is requirements.

The domain identification unit 230 may identify a domain corresponding to a specific activity for generating intervention information after the evaluation process. For example, the domain (i.e., a wellness domain) may be formed through a combination of various sub-domains such as a society, an environment, economy, education, and health.

The factor extractor 240 may extract a quantification factor corresponding to the identified domain, and for example, the quantification factor may be a factor corresponding to at least one of meal information, physical activity information, smoking information, or drinking information of the user.

The evaluation rule loading unit 240 may receive an appropriate guideline/rule from an expert data D/B for supporting evaluation of a specific activity using a specific context. In this case, the filtering process for receiving the appropriate instruction/rule may be based on a context and may disregard an instruction/rule with inconsistent contexts.

The life log accumulator 250 may accumulate behavior data corresponding to the extracted quantification factor of the life log data as the status of the activity of the user based on a corresponding expert instruction/rule during a preset accumulation time. For example, the life log accumulator 250 may accumulate data of an exercise amount (Phyexe) and a calorie consumption (Calbummin) using Equation 3 below.

$${Phy}_{exe} = {\sum }_{i = 1}^{n} \frac{\{ {exe}_{i} \}}{Week} \qquad [ Equation 3 ]$$

$${Cal}_{{burn}_{m i n}} = \frac{M E {T}_{exe} \times 3.5 \times Weight ( kg )}{200}$$

The lifestyle quantification unit 260 may quantify the accumulated behavior data (i.e., qualitative information of a behavior) based on a quantification rule preset by an expert.

For example, the lifestyle quantification unit 260 may calculate basal metabolic rate (BMR) information of the user based on the collected life log data and may quantify the accumulated behavior data by performing an operation on the calculated BMR information and an activity index corresponding to the accumulated data.

The lifestyle quantification unit 260 may calculate the BMI based on weight information and height information, which are collected in real time from the collected life log data.

A data collector of an intervention system according to an embodiment may collect behavior data quantified by the lifestyle quantification unit 260 and the calculated BMI as life log data.

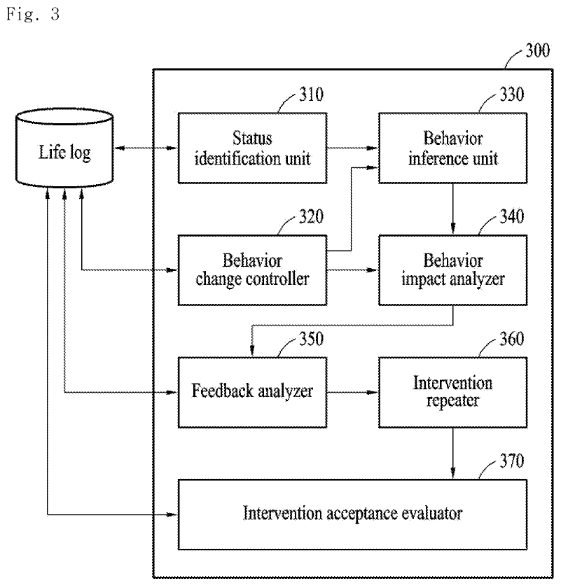

FIG. 3 is a diagram for explaining an intervention information controller according to an embodiment in detail.

Referring to FIG. 3, an intervention information controller 300 according to an embodiment may perform an intervention personalization process for generating user-customized intervention information based on a behavior status, a behavior change, and a behavior impact/adaptation status of the user, and an intervention evolution process for adjusting intervention information based on feedback information of the user to the intervention information that is generated through the intervention personalization process and is provided to the user.

In detail, the intervention personalization process may be performed through a status identification unit 310, a behavior change controller 320, a behavior inference unit 330, and a behavior impact analyzer 340, and the intervention evolution process may be performed through a feedback analyzer 350, an intervention repeater 360, and an intervention acceptance evaluator 370.

The status identification unit 310 may determine a health status of the user based on the quantified behavior data, and for example, the status identification unit 310 may determine the quantified behavior data as at least one health status of Healthy, Normal, or Risk based on a preset classification threshold.

The behavior change controller 320 may induce behavior change of the user, and may detect the behavior change of the user.

For example, the behavior change controller 320 may provide information corresponding to the quantified behavior data of the user of pre-stored initial intervention information to the user to induced behavior change and may detect the behavior change of the user.

The behavior inference unit 330 may infer a next level of a user behavior, which is to be achieved and executable, based on the determination result of the health status, the detection result of the behavior change of the user, and user preference and may generate intervention information based on inference result.

The behavior impact analyzer 340 may determine negative impact on the body of the user due to intervention information through monitoring of a biosignal, and when the determination result is negative, the behavior impact analyzer 340 may stop providing the generated intervention information.

In detail, the behavior impact analyzer 340 may monitor the negative impact based on the intervention information in consideration of an edmonton obesity staging system (EOSS) and may manage the intervention information to make an EOSS stage be a second stage or less.

The feedback analyzer 350 may evaluate implicit and explicit feedback of the user on the intervention provided to the user.

In detail, the feedback analyzer 350 may determine the number of times intervention information and a period at which the intervention information is provided based on a difference (Δ^{BMI}) between a body mass index (BMI_{End}) corresponding to the collected feedback information and a body mass index (BMIₛₜₐᵣₜ) corresponding to a time at which the generated intervention information is provided to the user.

For example, the feedback analyzer 350 may increase the number of interventions and/or may reduce a period at which intervention is provided (+ve) when the difference Δ_{BMI} between the received body mass indexes (BMIs) is equal to or greater than a preset body mass threshold, may not change the number of interventions and/or the period at which intervention is provided when there is no difference between the received BMIs (i.e., when the difference is 0), and may reduce the number of interventions and/or may increase the period at which intervention is provided (−ve) when the difference between the received BMIs is less than the body mass threshold.

The intervention repeater 360 may lastly determine whether the number of times intervention information is provided and a period at which the intervention information is provided, determined through the feedback analyzer 350, are applied in consideration of health status, body requirements, and physical ability of the user, which are derived from the collected life log data.

The intervention acceptance evaluator 370 may evaluate an acceptance level of intervention information that is subsequently provided through the intervention repeater 360 and may evaluate a degree by which the intervention information that is subsequently provided needs to be maintained.

For example, the intervention acceptance evaluator 370 may provide intervention information with one step lower than the previously provided intervention information when the difference Δ_{BMI} between BMIs is equal to or less than a first evaluation threshold, may provide intervention information with the same step as the previously provided intervention information when the difference Δ_{BMI} between BMIs is positioned between a preset first evaluation threshold and a second evaluation threshold (here, the second evaluation threshold>the first evaluation threshold), and may provide intervention information with one step higher than the previously provided intervention information when the difference Δ_{BMI} between BMIs is equal to or greater than the second evaluation threshold.

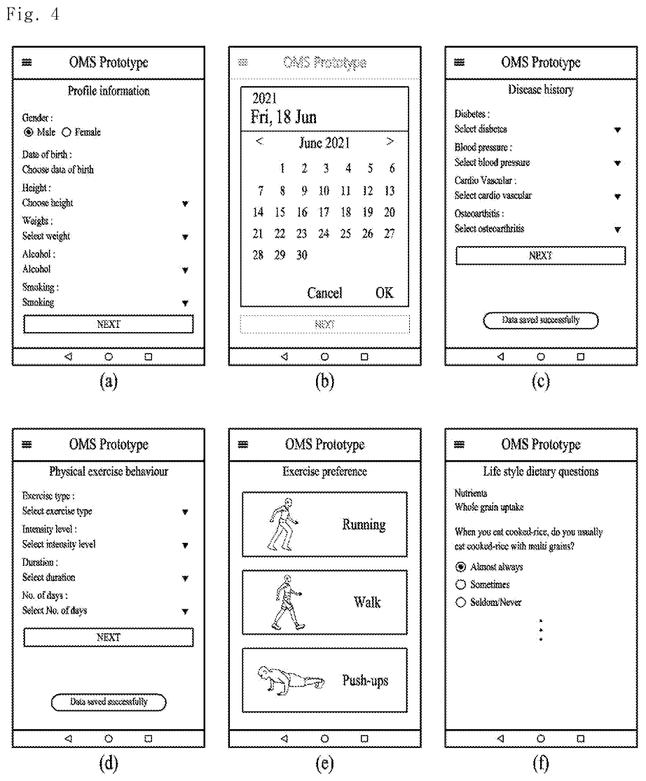

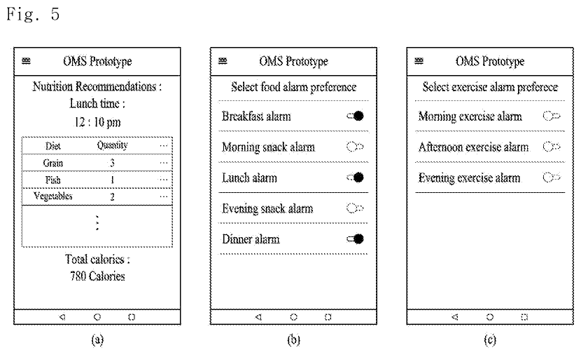

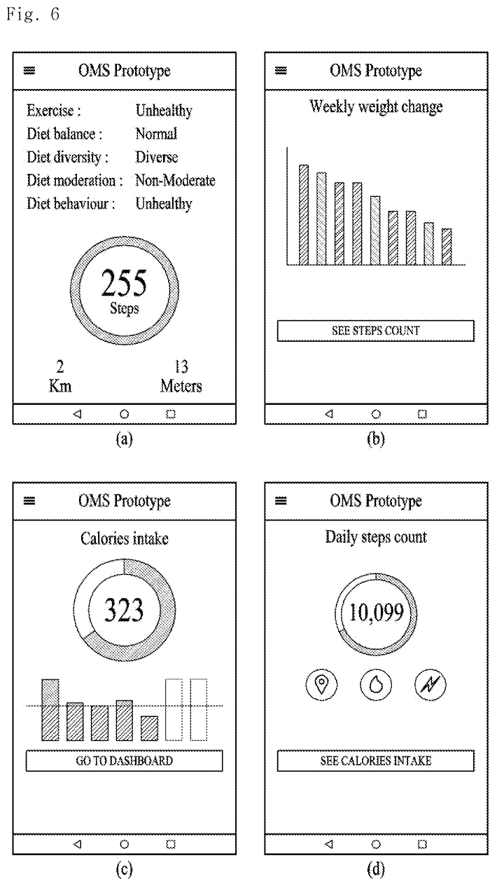

FIG. 4 is a diagram for explaining an example of collection of user information in a feedback-based intervention system according to an embodiment. FIGS. 5 and 6 are diagrams for explaining an example of intervention information provided in a feedback-based intervention system according to an embodiment.

Referring to FIGS. 4 to 6, the intervention system according to an embodiment may collect user information (i.e., profile data/life log data) through a preset application (App) and may provide intervention information, generated through analysis based on the collected data, to the user through the application.

As shown in (a)-(f) of FIG. 4, the intervention system may provide a survey for collecting the profile data/life log data through the application when the user accesses the intervention system, and may collect at least one of gender information, physical activity information, age information, height information, weight information, disease history information, blood pressure information, drinking information, smoking information, diet information, or meal information through user response to the survey.

Referring to (a)-(c) of FIG. 5, the intervention system may provide intervention information such as diet information, meal information/alarm, and exercise information/alarm, which are optimized for the user, to the user.

Referring to (a)-(d) of FIG. 6, the intervention system may visually provide information such as a behavior information status, a weight information status, a calorie intake status, and a step count status, which are derived during a procedure of generating the intervention information, to the user.

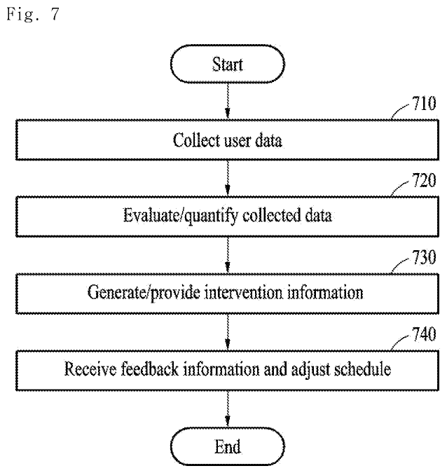

FIG. 7 is a diagram for explaining an operating method of a feedback-based intervention system according to an embodiment.

In other words, FIG. 7 is a diagram for explaining the operating method of the feedback-based intervention system according to an embodiment, which has been described above with reference to FIGS. 1 to 6, and hereinafter, with regard to a description with reference to FIG. 7, a repeated description of the description given with reference to FIGS. 1 to 6 will be omitted.

Referring to FIG. 7, in operation 710 of the operating method of the intervention system according to an embodiment, profile data and life log data of a user by a data collector may be collected.

Then, in operation 720 of the operating method of the intervention system according to an embodiment, at least one quantification factor may be extracted through an evaluation process based on profile data collected by a user data quantification unit and data corresponding to the extracted quantification factor of the collected life log data may be quantified.

Then, in operation 730 of the operating method of the intervention system according to an embodiment, a health status of the user may be determined based on the quantified behavior data and at least one piece of intervention information may be generated based on the determination result of the health status.

In operation 730 of the operating method of the intervention system according to an embodiment, intervention information generated by a communication unit may be provided to a user.

Then, in operation 740 of the operating method of the intervention system according to an embodiment, feedback information of the user, corresponding to the provided intervention information, may be received, and a provision schedule of the intervention information may be adjusted based on the received feedback information.

In conclusion, the present invention may provide a user-customized indication management service based on basic information of a user and behavior data based on a lifestyle of the user.

In addition, the present invention may provide a more effective and optimized indication management service to a user by applying user feedback for a provided indication management service.

An embodiment of the present invention may provide a user-customized indication management service based on basic information of a user and behavior data based on a lifestyle of the user.

An embodiment of the present invention may provide a more effective and optimized indication management service to the user by applying user feedback for the provided indication management service.

Although exemplary embodiments of the present invention have been disclosed for illustrative purposes, those skilled in the art will appreciate that various modifications, additions and substitutions are possible, without departing from the scope and spirit of the invention as disclosed in the accompanying claims. For example, a proper result may be achieved even if the techniques described above are implemented in an order different from that for the disclosed method, and/or disclosed constituents such as a system, structure, device and circuit are coupled to or combined with each other in a form different from that for the disclosed method or replaced by other constituents or equivalents.

It should be understood, however, that there is no intent to limit the invention to the embodiments disclosed, rather, the invention is to cover all modifications, equivalents, and alternatives falling within the spirit and scope of the invention as defined by the claims.

[Description of reference numerals]

| | |
|---|---|
| 100: intervention system | 110: data collector |
| 120: user data quantification unit 130: intervention information controller | 140: communication unit |

What is claimed is:

1. A feedback-based intervention system comprising:
a data collector configured to collect profile data and life log data of a user;
a user data quantification unit configured to extract at least one quantification factor through an evaluation process of the user based on the collected profile data, and to quantify behavior data of the collected life log data, which corresponds to the extracted quantification factor;
an intervention information controller configured to determine a health status of the user based on the quantified behavior data, and to generate at least one piece of intervention information based on a determination result of the health status; and
a communication unit configured to provide the generated intervention information to the user,
wherein the intervention information controller receives feedback information of the user, corresponding to the provided intervention information, and adjusts a provision schedule of the intervention information based on the received feedback information.

2. The feedback-based intervention system according to claim 1, wherein the profile data includes at least one of gender information, age information, initial height information, initial weight information, or disease history information of the user.

3. The feedback-based intervention system according to claim 1, wherein the life log data includes at least one of physical activity information, weight information, height information, blood pressure information, drinking information, smoking information, diet information, or meal information of the user, which is collected for a preset collection time.

4. The feedback-based intervention system according to claim 1, wherein the user data quantification unit calculates at least one piece of initial biological information of an initial obesity class, an initial body mass index (BMI), an initial calorie consumption, or an initial exercise amount of the user based on the collected profile data and extracts a quantification vector based on the calculated initial biological information.

5. The feedback-based intervention system according to claim 1, wherein the user data quantification unit accumulates behavior data corresponding to the extracted quantification factor of the collected life log data during a preset accumulation time and quantifies the accumulated behavior data based on a quantification rule preset by an expert.

6. The feedback-based intervention system according to claim 1, wherein the user data quantification unit quantifies the accumulated behavior data by calculating basal metabolic rate (BMR) information of the user based on the collected life log data and performing an operation on the calculated BMR information and an activity index corresponding to the accumulated behavior data.

7. The feedback-based intervention system according to claim 1, wherein the quantification factor is a factor corresponding to at least one of meal information, physical activity information, smoking information, or drinking information of the user.

8. The feedback-based intervention system according to claim 1, wherein the intervention information controller determines the quantified behavior data as at least one health status of ‘Healthy’, ‘Normal’, or ‘Risk’ based on a preset classification threshold, calculates behavior change information of the user based on the collected life log data, and calculates the at least one piece of intervention information based on a determination result of the health status and the calculated behavior change information.

9. The feedback-based intervention system according to claim 8, wherein the intervention information controller monitors biosignal information of the user, corresponding to the calculated behavior change information, and determines whether the generated intervention information is provided based on a monitoring result of the biosignal information.

10. The feedback-based intervention system according to claim 1, wherein the communication unit determines a provision schedule of the generated intervention information based on at least one of an eating habit of the user, an exercise routine of the user, an achievement goal of the user, preference of the user, or prescription of an expert.

11. The feedback-based intervention system according to claim 1, wherein the intervention information controller determines a number of times the intervention information is provided and a period at which the intervention information is provided based on a difference between a body mass index (BMI) corresponding to the collected feedback information and a BMI corresponding to a time at which the generated intervention information is provided to the user.

12. An operating method of a feedback-based intervention system, the method comprising:
collecting profile data and life log data of a user, by a data collector;
extracting at least one quantification factor through an evaluation process based on the collected profile data and quantifying data of the collected life log data, which corresponds to the extracted quantification factor, by a user data quantification unit;
determining a health status of the user based on the quantified behavior data and generating at least one piece of intervention information based on a determination result of the health status, by an intervention information controller;
providing the generated intervention information to the user, by a communication unit; and
receiving feedback information of the user, corresponding to the provided intervention information, and adjusting a provision schedule of the intervention information based on the received feedback information, by the intervention information controller.
